(19) United States
(12) Patent Application Publication
(10) Pub. No.: US 2004/0152896 A1
(43) Pub. Date: Aug. 5, 2004

(54) Process for the preparation of pyrrolidinyl ethylamine compounds via a copper-mediated aryl amination

(76) Inventors: Stephane Caron, Stonington, CT (US); Arun Ghosh, Madison, CT (US); Janice Ethel Sieser, Ivorytown, CT (US)

Correspondence Address: Warner-Lambert Company LLC, 2800 Plymouth Road, Ann Arbor, MI, 48105, US

(21) Appl. No.: 10/699,106

(22) Filed: Oct. 31, 2003

Related U.S. Application Data
(60) Provisional application No. 60/423,328, filed on Nov. 1, 2002.

(51) Int. Cl. C 07D 4 3/02

(52) U.S. Cl. 544/331; 546/276.4; 548/517; 548/527

(57) ABSTRACT

The invention provides a new process for the preparation of compounds of formula (XI):

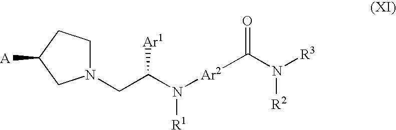

which are useful as kappa agonists. The process involves, as a key step, a copper-mediated aryl amination.

This United States Patent Application claims the benefit of priority to U.S. Application No. 60/423,328 filed Nov. 1, 2002.

The present invention relates to a new process for the preparation of pyrrolidinyl ethylamine compounds that comprises an efficient cuprous salt mediated aryl amination step.

The pyrrolidinyl ethylamine compounds that may be prepared in accordance with the process of the present invention (disclosed in U.S. Pat. No. 6,201,007) are compounds of formula (XI):

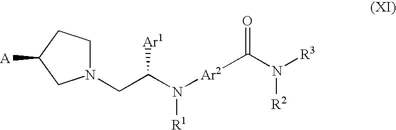

and stereoisomers thereof, wherein;

A is hydrogen, hydroxy, C₁-C₆ (preferably C₁-C₄) alkyl, C₁-C₆ (preferably C₁-C₄) fluoroalkyl (particularly —CF₃), C₁-C₆ (preferably C₁-C₄) alkoxy, or OY wherein Y is a hydroxy protecting group or A, taken together with its geminal hydrogen, is an oxo group;

Ar¹ is phenyl optionally substituted by one or more (preferably one to two) substituents selected from fluoro, C₁-C₄ alkyl, C₁-C₄ alkoxy, C₁-C₄ alkoxy-C₁-C₄ alkoxy, trifluoromethyl, carboxy-C₁-C₄ alkoxy and C₁-C₄ alkoxycarbonyl-C₁-C₄ alkoxy;

Ar² is phenyl, naphthyl, pyridyl, thienyl, furyl, pyrrolyl or pyrimidyl, each being optionally substituted by one or more (preferably one to two) substituents selected from fluoro, C₁-C₄ alkyl, C₁-C₄ alkoxy, di(C₁-C₄)alkylamino and C₁-C₄ fluoroalkyl;

R¹ is C₁-C₆ alkyl or benzyl wherein the phenyl moiety of said benzyl is optionally substituted with C₁-C₆ alkoxy or OY wherein Y is a hydroxy protecting group; and

R² and R³ are independently selected from hydrogen, C₁-C₇ alkyl optionally substituted by one or more (preferably one to five) hydroxy or halo groups, C₃-C₆ cycloalkyl, C₂-C₆ alkenyl, C₂-C₆ alkynyl, C₁-C₇ (preferably C₁-C₅) alkoxy, phenyl optionally substituted by fluoro (preferably substituted by one or two fluoro groups), phenyl-C₁-C₇ (preferably C₁-C₅) alkyl wherein the phenyl group is optionally substituted by fluoro, and —(CH₂)ₙX—R⁴ wherein n is one or two, X is O or S and R⁴ is C₁-C₃ alkyl, or, when Ar² is phenyl, —Ar²—C(═O)—N(R²)— is a phthalimide group and R is C₁-C₇ alkyl; or

R² and R³, together with the nitrogen atom to which they are attached, form a pyrrolidine, piperidine or morpholine ring, optionally substituted by C₁-C₃ alkyl or fluoro.

When Ar² is phenyl, R²R³N—C(═O)— is preferably at the meta or para position on the phenyl ring with respect to 2-(A-pyrrolydinyl)-1-Ar¹-ethyl-N(R¹)—.

As disclosed in U.S. Pat. No. 6,201,007, these compounds are selective kappa receptor agonists and hence useful in the treatment of a variety of diseases, particularly as analgesic, anesthetic, anti-inflammatory and neuroprotective agents.

The route described in U.S. Pat. No. 6,201,007 for making the compounds of formula (XI) utilizes styrene oxide which is a dangerous material to work with. Furthermore, the route does not lend itself to an efficient stereoselective synthesis since stereocontrol is poor and diastereomeric intermediates are formed which are difficult to separate.

There is therefore a need to provide a new, efficient, short and high-yielding synthesis of compounds of the formula (XI) which does not suffer from the disadvantages of the prior art process. Such a synthesis is unexpectedly provided by the process of the present invention which is described in detail below. One of the key steps in the new process is a copper-catalysed coupling between an aryl halide and an oxazolidinone. The use of this step has provided unexpected advantages being surprisingly high-yielding, mild, efficient, cost-effective and robust.

A compound of formula (XI), as defined above, or a stereoisomer thereof, may be prepared in accordance with the present process by the reductive alkylation of a compound of formula (X):

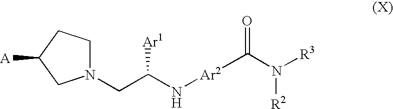

wherein A, Ar¹, Ar², R² and R³ are as defined above, or a stereoisomer thereof.

Reductive alkylation with an aldehyde alkylating agent and a boron hydride reducing agent is preferred, with decaborane most preferred as the reducing agent.

In a further aspect of the present invention, a compound of formula (X), or a stereoisomer thereof, wherein A, Ar¹, Ar², R² and R³ are as defined above, may be prepared by hydrolytically cleaving the —SO₃H group in a compound of formula (IX):

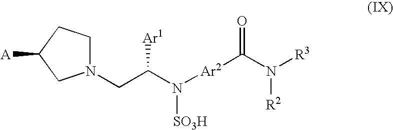

or a zwitterion thereof, or a stereoisomer of either, wherein A, Ar¹, Ar², R² and R³ are as defined above, preferably with a strong acid, most preferably with a mineral acid.

In a further aspect of the invention a compound of formula (IX), or a zwitterion thereof, or a stereoisomer of either, may be prepared by treating a compound of formula (VII):

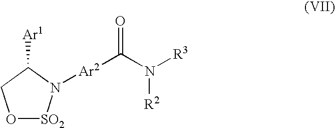

wherein Ar¹, Ar², R² and R³ are as defined above, or the enantiomer thereof, with a compound of formula (VIII):

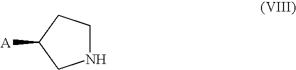

wherein A is as defined above, or the enantiomer thereof.

In a further aspect of the invention, a compound of formula (VII), wherein Ar¹, Ar², R² and R³ are as defined above, or the enantiomer thereof, may be prepared by oxidising a compound of formula (VI):

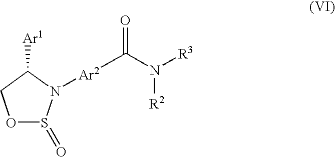

wherein Ar¹, Ar², R² and R³ are as defined above, or the enantiomer thereof. The oxidation is preferably carried out with a mixture comprising a compound selected from ruthenium trichloride, ruthenium tribromide or ruthenium triiodide and hydrates thereof, preferably ruthenium trichloride and hydrates thereof, and a compound selected from sodium periodate (NaIO₄), potassium permanganate (KMnO₄), sodium hypochlorite (NaOCl) and potassium periodate (KIO₄), preferably NaIO₄.

In a further aspect of the invention, a compound of formula (VI) or the enantiomer thereof, wherein Ar¹, Ar², R² and R³ are as defined above, may be prepared by treating a compound of formula (V):

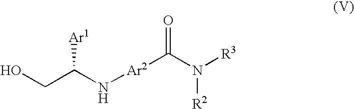

or the enantiomer thereof, wherein Ar¹, Ar², R² and R³ are as defined above, with a thionyl halide, preferably SOCl₂ or SOBr₂, most preferably SOCl₂.

In a further aspect of the invention a compound of formula (V), or the enantiomer thereof, wherein Ar¹, Ar², R² and R³ are as defined above, may be prepared by treating a compound of formula (IV):

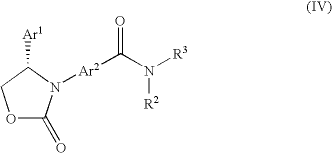

or the enantiomer thereof, wherein Ar¹, Ar², R² and R³ are as defined above with a base in the presence of water. The base is preferably sodium hydroxide, potassium hydroxide, lithium hydroxide, cesium hydroxide or a quaternary ammonium hydroxide such as n-tert-butylammonium hydroxide (n-Bu₄NOH), preferably sodium hydroxide.

In a further aspect of the invention, a compound of formula (IV), or the enantiomer thereof, wherein Ar¹, Ar², R² and R³ are as defined above, may be prepared by treating a compound of formula (II):

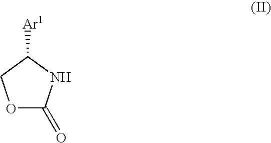

or the enantiomer thereof, wherein Ar¹ is as defined above, with a compound of formula (III):

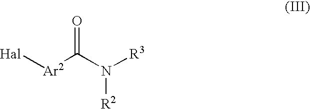

wherein Ar², R² and R³ are as defined above and wherein one unsubstituted position on the Ar² moiety is substituted with a halogen group Hal, preferably chloro (Cl), bromo (Br) or iodo (I), most preferably Br, in the presence of a cuprous salt, an amino ligand and a base. The cuprous salt is preferably copper (I) iodide (CuI), copper (I) bromide (CuBr) or copper (I) chloride (CuCl), most preferably, CuI. The amino ligand is preferably a chelating ligand, most preferably 1,2-diaminocyclohexane.

The base is preferably sodium carbonate, potassium carbonate or cesium carbonate, most preferably potassium carbonate.

In a further aspect of the invention, a compound of formula (II), or the enantiomer thereof, wherein Ar¹ is as defined above, may be prepared by treating a compound of formula (I):

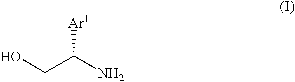

or the enantiomer thereof, wherein Ar¹ is as defined above, with a C₁-C₆ dialkyl carbonate, preferably diethylcarbonate, in the presence of a base. The base is preferably sodium carbonate, potassium carbonate or cesium carbonate, most preferably potassium carbonate.

The term “halo”, as used herein, unless otherwise indicated, means chloro, bromo or iodo.

The term “alkyl”, as used herein, unless otherwise indicated, means a saturated monovalent hydrocarbon radical having straight or branched moieties.

The term “alkoxy”, as used herein, means an —O-alkyl group wherein “alkyl” is defined above.

The term “aryl”, as used herein, unless otherwise indicated means an organic radical derived form an aromatic hydrocarbon by removal of one hydrogen, such as phenyl or naphthyl.

The term “C₁-C₆ alkyl” is used herein to mean a straight or branched alkyl including but not limited to methyl, ethyl, n-propyl, iso-propyl, n-butyl, sec-butyl, tert-butyl and the like.

The term “C₁-C₆ alkoxy” is used herein to mean a straight or branched —OR wherein R is C₁-C₆ alkyl, including, but not limited to, methoxy, ethoxy, propoxy, isopropoxy, n-butoxy, iso-butoxy, tert-butoxy and the like.

The term “halo C₁-C₆ alkyl” means a straight or branched, halo-substituted alkyl of 1 to 6 carbon atoms including, but not limited to methyl, ethyl, n-propyl, iso-propyl, n-butyl, sec-butyl and tert-butyl, substituted by 1 to 13 (preferably one to five) halogen atoms.

The term “halo C₁-C₆ alkoxy” means C₁-C₆ alkoxy substituted by 1 to 13 (preferably one to three) halogen atoms.

The term “halo substituted phenyl C₁-C₇ alkyl” means C₁-C₇ alkyl having a phenyl group attached to its terminal carbon atom, the phenyl group being substituted by one to five (preferably one to two) halogen atoms.

In this specification, the term “hydroxy protecting group” means a functional group to protect a hydroxy group against undesirable reactions during synthetic procedures, including, but not limited to benzyl, benzoyl, methoxymethyl, tetrahydropyranyl and trialkylsilyl. Suitable groups are described in ‘Protective Groups in Organic Synthesis’ by Theorora Greene and Peter Wuts (third edition, 1999, John Wiley and Sons).

The term ‘stereoisomer’ means an enantiomer or diastereomer, which have the normal meaning ascribed to them in the art. For instance, the stereoisomers of a compound of formula (XI), as defined above, include its enantiomer (XIa) and its diastereomeric forms (XIb) and (XIc).

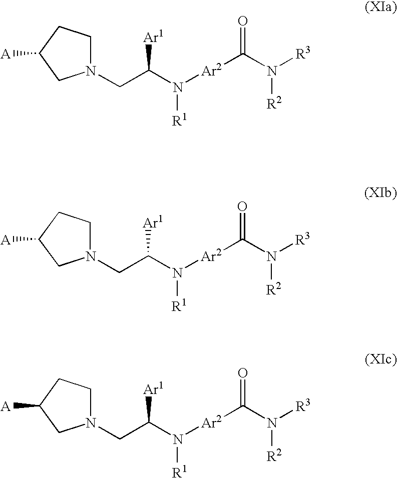

It should be noted that in certain circumstances, when A is hydrogen or, together with its geminal hydrogen, is oxo, only enantiomers will exist.

The processes of the present invention are illustrated in Schemes 1 and 2, in which, unless otherwise indicated, A, R¹, R², R³, Ar¹, Ar² and Hal are as defined above.

Overall, the synthetic sequence (Schemes 1 and 2) involves forming an oxazolidinone (II) from compound (I) with an alkyl carbonate (step 1), Cu¹ mediated coupling of oxazolidinone (II) with halo substituted arylamide (III) to form N-substituted oxazolidinone (IV) (step 2), hydrolytic decarbonylation of the oxazolidinone ring of N-arylated oxazolidinone (IV) under basic conditions to give 2-hydroxy-1-aryl-ethylamino arylamide (V) (step 3), conversion of 2-hydroxy-1-aryl-ethylamino arylamide (V) to N-substituted 2-oxo-4-aryl-[1,2,3]oxathiazolidine (VI) via reaction with a thionyl halide (step 4), oxidation of N-substituted 2-oxo-4-aryl-[1,2,3]oxathiazolidine (VI) to 2,2-dioxo-4-aryl-[1,2,3]oxathiazolidine (VII) (step 5), ring opening nucleophilic displacement of sulfonate by treatment of 2,2-dioxo-4-aryl-[1,2,3]oxathiazolidine (VII) with pyrrolidinyl derivative (VIII) in the presence of base to form sulfamic acid (IX) (step 6), acid hydrolysis of the intermediate sulfamic acid to remove sulfonate and produce compound (XI) wherein R¹ is H (step 7), and reductive alkylation to produce compound (XI) wherein R¹ is C₁-C₆ alkyl or benzyl wherein the phenyl moiety of said benzyl is optionally substituted with C₁-C₆ alkoxy or OY wherein Y is a hydroxy protecting group (step 8).

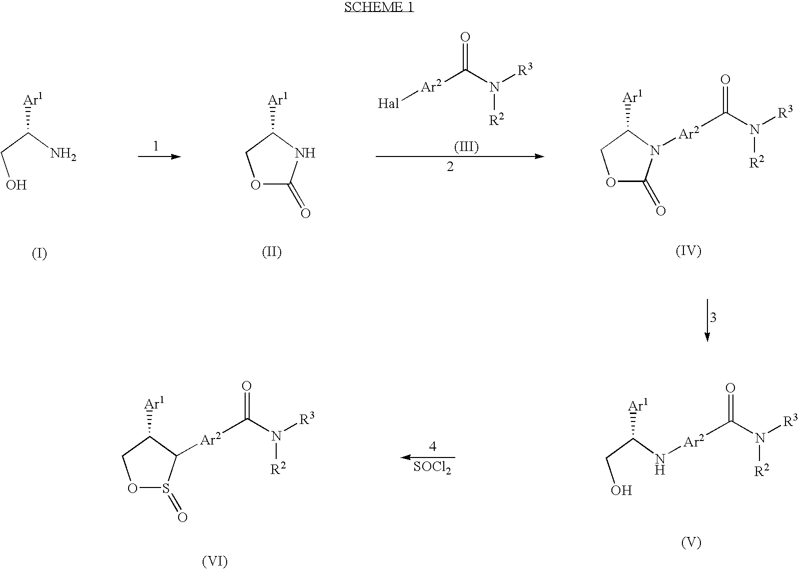

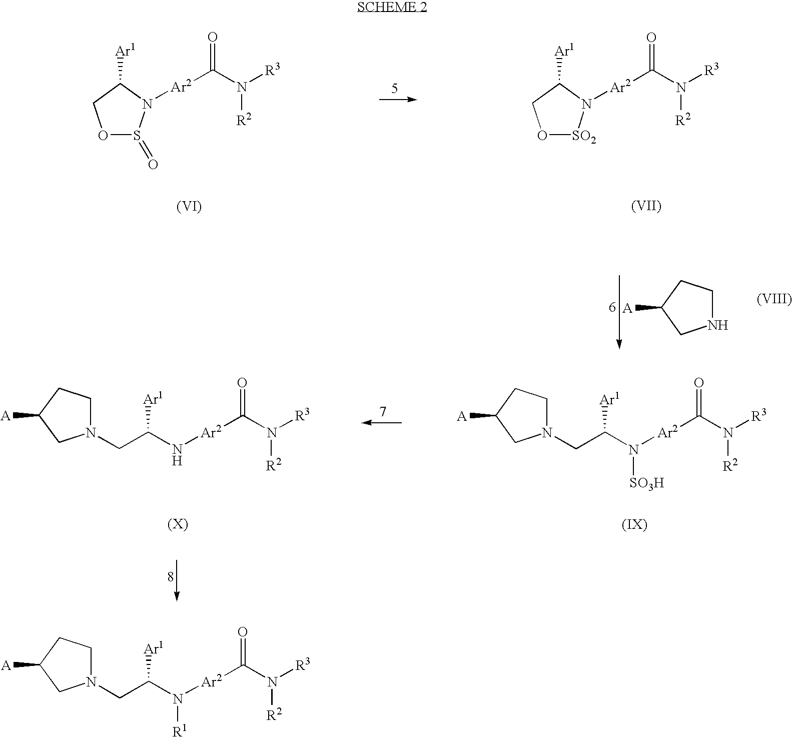

In step 1 of Scheme 1 chiral arylglycinol of formula (I), or its enantiomer, wherein Ar¹ is as defined above, is treated under conditions known in the art with a C₁-C₆ alkyl carbonate, preferably diethylcarbonate, in the presence of a base selected from sodium carbonate, potassium carbonate and cesium carbonate, preferably potassium carbonate, to form compound (II).

Step 2 of Scheme 1 is a copper catalyzed N-arylation of compound (II) or its enantiomer. This approach, which is a variation of the classical Goldberg/Ullmann approach is discussed in Kiapars, et al., J. Am. Chem. Soc. (2001), 123(31), 7727-7729 and Ma, et al., Org. Lett. (2001), 3(16), 2583-2586. In the context of the present process the copper-mediated N-arylation provides unexpected and outstanding advantages. In particular, it is:

high-yielding compared to other coupling reactions tested; mild and efficient, not requiring high-temperatures or stoichiometric amounts of metal;

cost-effective, not requiring the use of an expensive metal such as palladium; and

insensitive to the presence of functional groups in the coupling partners.

Oxazolidinone (II) wherein Ar¹ is as defined above, or the enantiomer thereof, and halo substituted arylamide (III) wherein Ar², R² and R³ are as defined above, are mixed with a cuprous salt selected from CuI, CuBr and CuCl, preferably CuI and a base selected from sodium carbonate, potassium carbonate and cesium carbonate, preferably potassium carbonate, in an inert atmosphere preferably comprising nitrogen or argon. An aprotic solvent, preferably an ethereal solvent having a relatively high boiling point, most preferably dioxane, is added to the mixture held under an inert atmosphere. While maintaining the reaction mixture under an inert atmosphere, an amino ligand, preferably a diamino chelating ligand, most preferably 1,2 diaminocyclohexane, is added in approximately equimolar amount to the Cu¹ in the reaction mixture. The reaction mixture is heated to from about 100° C. to about 120° C., preferably to about 110° C., for a period of from about 12 hours to about 17 hours, preferably from about 15 hours to about 16 hours, giving N-arylated oxazolidinone (IV) or its enantiomer.

Step 3 of Scheme 1 is a hydrolytic decarbonylation of the oxazolidinone ring of N-arylated oxazolidinone (IV), or its enantiomer, under basic conditions using a base selected from lithium hydroxide (LiOH), sodium hydroxide (NaOH) and potassium hydroxide (KOH), preferably NaOH, to give 2-hydroxy-1-aryl-ethylamino arylamide (V), or its enantiomer. The decabonylation of compound (IV) is preferably conducted in a hydroxylic solvent such as water or a C₁-C₆ alcohol, most preferably ethanol, although ethereal solvents and mixtures of ethereal and hydroxylic solvents may also be used, at a temperature of from about 40° C. to about 60° C., preferably at about 50° C., for a period of from about 10 minutes to about 60 minutes, preferably from about 15 minutes to about 25 minutes.

The resultant reaction mixture is preferably concentrated and diluted with water followed by extraction with an organic solvent such as a chlorinated hydrocarbon, preferably dichloromethane, to obtain aminol (V).

Step 4 of Scheme 1 is the conversion of aminol (V), or its enantiomer, to N-substituted 2-oxo-4-aryl-[1,2,3]oxathiazolidine (VI), or its enantiomer, via reaction with a thionyl halide, preferably thionyl chloride. The reaction is carried out in the presence of a base such as a tertiary amine, preferably pyridine, in a non-hydroxylic solvent, preferably an ethereal solvent such as tetrahydrofuran (THF), diisopropyl ether or methyl tert-butyl ether, most preferably THF. The reaction is commenced at an initial temperature below ambient temperature, preferably at about 0° C., followed by slow warming over a period of from about 1 hour to about 18 hours, preferably from about 12 to about 16 hours, to about ambient temperature.

Step 5 of Scheme 2 is the oxidation of N-substituted 2-oxo-4-aryl-1,2,3]oxathiazolidine (VI), or its enantiomer, to 2,2-dioxo-4-aryl-[1,2,3]oxathiazolidin-3-yl-arylamide (VII), or its enantiomer, in an organic solvent or solvent mixture, preferably a non-oxygenated solvent or solvent mixture, most preferably a mixture of dichloromethane and acetonitrile (CH₃CN), by treatment with a ruthenium trihalide, preferably RuCl₃, and a periodate salt, preferably NaIO₄, preferably at about 0° C., for from about 30 to about 70 minutes, preferably for from about 45 to about 55 minutes. The oxidation of step 5 may also be carried out with other oxidation agents well known in the art such as KMnO₄, or other permanganate salts, in a solvent such as an acetic acid/water mixture; or with NaOCl or another hypochlorite salt in an organic solvent such as CH₃CN.

In step 6 of Scheme 2 pyrrolidinyl derivative (VIII), wherein A is as defined above, or the enantiomer thereof, is used to effect a ring opening nucleophilic displacement of the sulfonate moiety of 2,2-dioxo-4-aryl-[1,2,3]oxathiazolidine (VII), or the enantiomer thereof, resulting in N-substitution of pyrrolidinyl derivative (VIII) to produce a sulfamic acid having the formula (IX), or the zwitterion thereof, or a stereoisomer of either. The 2,2-dioxo-4-aryl-[1,2,3]oxathiazolidine (VII) is treated with an excess of the pyrrolidinyl derivative (VIII), the molar ratio of the pyrrolidinyl derivative (VIII) to the 2,2-dioxo-4-aryl-[1,2,3]oxathiazolidine (VII) typically being from about 1.5:1 to about 2.5:1 with a molar ratio of about 2.35:1 preferred. A tertiary amine base such as triethylamine or diisopropylethylamine, preferably triethylamine, is also added to the reaction mixture, the molar ratio of the tertiary amine base to the pyrrolidinyl derivative (VIII) being from about 0.8:1 to about 1.2:1, with a molar ratio of about 1:1 preferred. The reaction is carried out at a temperature of from about 20° C. to about 25° C. for from about 2 hours to about 18 hours, preferably for from about 14 to about 16 hours. The intermediate sulfamic acid having the formula (IX) is then freed of amine.

In step 7 of Scheme 2 the —SO₃H group of compound (IX), or a stereoisomer thereof, is hydrolytically removed by heating compound (IX), or a stereoisomer thereof, in the presence of an acid, preferably a strong acid, more preferably a mineral acid, for from about 1 to about 3 hours, preferably for from about 1.5 to about 2.5 hours, at a temperature of from about 40° C. to about 60° C., preferably from about 45° C. to about 55° C., to yield compound (X), or a stereoisomer thereof.

In step 8 of Scheme 2, compound (X), or a stereoisomer thereof, is reductively alkylated to produce compound (XI), or a stereoisomer thereof, wherein R¹ is C₁-C₆ alkyl, by treatment with a C₁-C₆ aldehyde and a reducing agent, preferably a boron hydride, more preferably decaborane, wherein each mole of compound (XI) is treated with said aldehyde and said boron hydride in a molar ratio of aldehyde to boron hydride of about 5 to about 1 preferably about 4.4 to about 1 at a temperature of from about 20° C. to about 25° C. for about from 20 hours to about 140 hours, with from about 110 to about 130 hours preferred. An approximately 25% to about 30% portion of the initial amount of aldehyde and an approximately 25% to about 30% portion of the initial amount of hydride reducing agent may be optionally added to the reaction mixture after from about 22 to about 26 hours and after from about 46 to about 50 hours. Compound (X) wherein R¹ is benzyl or substituted benzyl is prepared from benzaldehyde or a substituted benzaldehyde under similar conditions. Optionally, compound (XI) having hydroxy protecting group Y is treated to remove group Y.

The present invention is illustrated by the following examples, but it is not limited to the details thereof. In the following examples, the term “ambient temperature” means a temperature of from about 20° C. to about 25° C. The following abbreviations are used: HPLC, high performance liquid chromatography; MS, mass spectroscopy; NMR, nuclear magnetic resonance; TLC, thin layer chromatography.

### EXAMPLE 1

4-(2-Oxo-4-phenyl-oxazolidin-3-yl)-N-propyl-benzamide

S-(+)-4-Phenyl-oxazolidin-2-one (2.81 g, 17.2 mmol), 4-bromo-N-propyl-benzamide (4.17 g, 17.2 mmol), CuI (0.32 g, 1.72 mmol) and potassium carbonate (4.76 g, 17.2 mmol) were charged to a nitrogen-purged flask. The flask was evacuated and backfilled with nitrogen before addition of dioxane (17.2 ml). To the above reaction mixture, 1,2-diaminocyclohexane (0.21 ml, 1.72 mmol) was added via syringe. The resulting bright blue mixture was heated at 110° C. for 15.5 hours. Analysis (HPLC/MS) of the reaction mixture indicated that the reaction was complete.

The oil bath was cooled to 45° C., and any precipitated product was dissolved by the addition of dichloromethane (50 ml). The mixture was filtered through celite and the solids were washed with an additional 50 ml of warm dichloromethane. The combined filtrates were concentrated and vacuum dried to give the desired oxazolidine as a light brown solid in near quantitative yield (5.6 g).

Mass spec: 325 (m+1).

¹H NMR (CDCl₃) δ 0.94 (t, J=7.5, 3H), 1.59 (m, 2H), 3.35 (m, 2H), 4.21 (m, 1H), 4.80 (m, 1H), 5.43 (m, 1H), 6.16 (br, 1H), 7.27 (d, J=7.9, 2H), 7.34 (m, 3H), 7.46 (d, J=8.0, 2H), 7.64 (d, J=8.3, 2H).

¹³C NMR (CDCl₃) δ 11.66, 23.08, 41.96, 60.54, 70.10, 120.10, 126, 127.95, 129.24, 129.75, 130.77, 137.96, 139.81, 155.84, 167.02.

### EXAMPLE 2

4-(2-Hydroxy-1-phenyl-ethylamino)-N-propyl-benzamide

A suspension of the oxazolidinone of Example 1 (3.025 g, 10 mmol) in 12.5 N sodium hydroxide (4 ml, 50 mmol) and ethanol (33 ml) was heated to 50° C. The reaction was complete within 20 minutes as observed by HPLC/MS analysis. The reaction mixture was concentrated and water (20 ml) followed by dichloromethane (30 ml) were added to the dark residue. After separation of the phases, the aqueous layer was extracted three times with 15 ml portions of dichloromethane. The combined organic layers were washed with brine and dried over sodium sulphate. Concentration and granulation in ethyl ether gave a 70% recovery of the title aminol as a light brown solid (2.15 g).

Mass spec: 299 (m+1).

¹H NMR (CDCl₃) δ 0.90 (t, J=7.5, 3H), 1.55 (m, 2H), 3.30 (m, 2H), 3.78 (m, 1H), 3.91 (m, 1H), 4.46 (m, 1H), 6.47 (d, J=8.3, 2H), 7.25 (m, 5H), 7.42 (d, J=8.7, 2H).

¹³C NMR (CDCl₃) δ 11.68, 23.12, 41.97, 61.29, 66.86, 114.32, 124.03, 127.13, 128.06, 128.52, 129.04, 139.03, 149.11, 168.29.

### EXAMPLE 3

4-(2-Oxo-4-phenyl-[1,2,3]oxathiazolidin-3-yl)-N-propyl-benzamide

Thionyl chloride (0.33 ml, 4.5 mmol) and anhydrous pyridine (4.3 ml, 53.5 mmol) were added to anhydrous tetrahydrofuran (4.7 ml) chilled in an ice bath. 4-(2-Hydroxy-1-phenyl-ethylamino)-N-propyl-benzamide (0.64 g, 2.1 mmol) dissolved in 30 ml anhydrous tetrahydrofuran was added via addition funnel over 30 minutes to the rapidly stirred ice-cold mixture. The mixture was stirred overnight, warming slowly to ambient temperature.

Water (15 ml) and methyl tert-butyl ether (15 ml) were added to the reaction mixture and the organic layer was separated. The aqueous layer was extracted twice with 15 ml methyl tert-butyl ether. The combined organic extracts were washed with brine, dried over K₂CO₃, filtered, and concentrated. The product was isolated as an off-white foam in 89% yield (0.643 g).

¹H NMR data indicated almost exclusively trans isomer (relationship of phenyl to the S═O) when the spectrum was obtained shortly after sample preparation in CDCl₃. Interestingly, after a few hours at room temperature, the same sample produced an equilibrated mixture of isomers (1:1).

Mass spec: 345 (m+1).

¹H NMR (CDCl₃) δ 0.928 (t, J=7.5, 3H), 1.57 (m, 2H), 3.34 (m, 2H), trans: 4.52 (m, 1H), 5.29 (m, 1H), 5.36 (m, 1H), 6.99 (m, 2H), 7.24 (m, 2H), 7.32 (m, 3H), 7.61 (m, 2H). cis: 4.89 (m, 1H), 5.00 (m, 1H), 5.15 (m, 1H), 6.98 (m, 2H), 7.33 (m, 3H), 7.46 (m, 2H), 7.61 (m, 2H).

³C NMR (CDCl₃) δ 11.65, 23.14, 41.91, 61.40, 64.13, 76.80, 78.05, 115.98, 117.88, 126.46, 127.41, 128.56, 128.9, 129.6, 134.48, 137.08, 142.02, 166.90.

### EXAMPLE 4

4-(2,2-Dioxo-4-phenyl-[1,2,3]oxathiazolidin-3-yl)-N-propyl-benzamide

The oxathiazolidine of Example 3, (0.11 g, 0.3 mmol) was dissolved in a mixture of dichloromethane and acetonitrile (1:1, 1.2 ml). The yellow solution was chilled in an ice-bath before addition of RuCl₃.H₂O (5 mg, 0.02 mmol), NaIO₄ (0.10 g, 0.46 mmol) and water (0.5 ml). HPLC/MS indicated the reaction was complete within 50 minutes. After warming to ambient temperature, the mixture was filtered through Celite™. The filtered solids were washed with methyl tert-butyl ether. The combined filtrates were washed with water and brine, then dried over sodium sulphate, filtered and concentrated. The product was recovered as an amber oil (79%, 0.085 g).

Mass spec: 361 (m+1).

¹H NMR (CDCl₃) δ 0.91 (t, J=7.5, 3H), 1.56 (m, 2H), 3.32 (m, 2H), 4.48 (dd, J=8.7, 1H), 4.94 (dd, J=8.9, 1H), 5.40 (dd, J=7.0, 1H), 7.14 (d, J=8.7, 2H), 7.35 (m, 5H), 7.62 (d, J=8.7, 2H).

¹³C NMR (CDCl₃) δ 11.64, 23.05, 41.99, 62.40, 73.07, 119.96, 126.94, 128.57, 129.79, 129.84, 131.66, 134.68, 138.39, 166.84.

### EXAMPLE 5

Benzoic acid 1-[2-phenyl-2-(4-propylcarbamoyl-phenylamino)-ethyl]-pyrrolidin-3-yl ester

Triethylamine (0.065 ml, 0.47 mmol) was added to a slurry of benzoic acid pyrrolidin-3-yl-ester hydrochloride (0.106 g, 0.47 mmol) and the dioxo oxathiazolidine of Example 4 (0.085 g, 0.2 mmol) in ethanol (2 ml). The mixture was stirred overnight at ambient temperature. HPLC/MS indicated only a trace of starting material remained. Excess amine was removed by washing a solution of the reaction mixture and ethyl acetate with dilute hydrochloric acid (0.5 mM). A water wash removed triethylamine hydrochloride from the mixture. The remaining ethyl acetate solution was heated to 50° C. with 1 N HCl (1 ml) for approximately 2 hours, cooled to ambient temperature and washed with a saturated brine solution. The aqueous layer was extracted twice with ethyl acetate and the combined extracts were dried over sodium sulphate. Filtration and concentration gave the product as an off-white foam (0.061 g, 66% yield).

Mass spec: 472 (m+1).

¹H NMR (CDCl₃) δ 0.91 (m, 3H), 1.55 (m, 2H), 2.05 (m, 1H), 2.37 (m, 1H), 2.66 (m, 2H), 2.85 (m, 1H), 2.97 (m, 2H), 3.11 (m, H1), 3.32 (m, 2H), 4.36 (m, 1H), 5.42 (m, 1H), 6.49 (d, J=7.5, 2H), 7.2-7.6 (m, 10H), 8.05 (m, 2H).

¹³C NMR (CDCl₃) δ 11.69, 23.26, 32.09, 41.75, 52.56, 56.80, 59.92, 62.72, 74.63, 113.43, 123.60, 126.49, 127.80, 128.44, 128.68, 129.10, 129.85, 133.42, 141.76, 150.62, 166.59, 167.63.

### EXAMPLE 6

Benzoic acid 1-{2-[methyl-(4-propylcarbamoyl-phenyl)-amino]-2-phenyl-ethyl}-pyrrolidin-3-yl ester

Benzoic acid 1-[2-phenyl-2-(4-propylcarbamoyl-phenylamino)-ethyl]-pyrrolidin-3-yl ester (0.21 g, 0.44 mmol) was dissolved in 2 ml methanol at ambient temperature. Aqueous formaldehyde solution (37% by weight, 0.07 ml, 0.88 mmol) was added followed by decaborane (0.025 g, 0.2 mmol). The mixture was stirred at ambient temperature for five days. Additional formaldehyde solution (0.02 ml each time) and decaborane (0.006 g each time) were added after 24 h and 48 h. The mixture was concentrated to a yellow residue and purified by column chromatography to isolate benzoic acid 1-{2-[methyl-(4-propylcarbamoyl-phenyl)-amino]-2-phenyl-ethyl}-pyrrolidin-3-yl ester in 50% yield (0.11 g). HPLC, MS and NMR analysis of the colorless oil showed it to be identical to a standard sample of benzoic acid 1-{2-[methyl-(4-propylcarbamoyl-phenyl)-amino]-2-phenyl-ethyl}-pyrrolidin-3-yl ester.

### EXAMPLE 7

4-(2-Hydroxy-1-phenyl-ethylamino)-N-propyl-benzamide

A suspension of the oxazolidinone of Example 1 (0.5 g, 1.54 mmol, 1.0 equivalent) in 12.5 N NaOH (0.19 ml, 2.38 mmol, 1.5 equivalent) and ethanol (1 ml) was heated to 50° C. The reaction was complete within 20 to 30 minutes as observed by TLC (75% ethyl acetate/25% hexanes) and HPLC/MS analysis. The reaction mixture was concentrated and the solid residue was treated with water (7 ml) and then dichloromethane (0.5 ml). The resulting mixture was stirred for four hours at ambient temperature. The fluffy residue was filtered and dried under vacuum to obtain the desired product as a white solid (441 mg, 96%).

### EXAMPLE 8

4-(2-Oxo-4-phenyl-[1,2,3]oxathiazolidin-3-yl)-N-propyl-benzamide

Thionyl chloride (11.0 ml, 151.2 mmol, 1.5 equivalents) and anhydrous pyridine (33.4 ml, 413.0 mmol, 4.1 equivalents) were added to anhydrous tetrahydrofuran (156 ml) chilled in an ice bath. 4-(2-Hydroxy-1-phenyl-ethylamino)-N-propyl-benzamide (30 g, 100.5 mmol) dissolved in anhydrous tetrahydrofuran (600 ml) was added via addition funnel over 3 hours to the rapidly stirred, ice-cold mixture. The reaction was nearly complete within 5 minutes (HPLC/MS analysis). The mixture was stirred overnight, warming slowly to ambient temperature.

Water (325 ml) and methyl tert-butyl ether (160 ml) were added to the reaction mixture and the organic layer was separated. The aqueous layer was extracted twice with methyl tert-butyl ether (130 ml). The combined organic extracts were washed with brine, dried over K₂CO₃, filtered and concentrated. The product was isolated as an off-white solid (33.6 g, 97%).

### EXAMPLE 9

4-(2,2-Dioxo-4-phenyl-[1,2,3]oxathiazolidin-3-yl)-N-propyl-benzamide

The trans-oxathiazolidine of Example 8, (0.0752 g, 0.218 mmol) was dissolved in dichloromethane (0.44 ml) and chilled in an ice-bath. To the above solution, 0.44 ml (0.04 M, 0.018 mmol, 0.08 equivalents) of a solution of RuCl₃.H₂O was added. The reaction mixture was stirred for 5 minutes and NaIO₄ (0.072 g, 0.339 mmol, 1.6 equivalents) was added followed by 0.36 ml of a buffer solution (pH 6.5). The reaction mixture was stirred vigorously. HPLC/MS indicated the reaction was complete within 10 minutes. After warming to ambient temperature, the mixture was filtered through Celite™. The filtered solids were washed with methyl tert-butyl ether. The combined filtrates were washed with water and brine, dried over sodium sulphate, filtered and concentrated. The product was recovered as a white solid (0.073 g, 92%).

HPLC/MS Conditions and Results

| | |
|---|---|
| Instrument: | Hewlett-Packard 1100 series HPLC/MS |
| Column: | Zorbax SB-CN 150 × 4.6 mm |
| Mobile Phase: | acetonitrile/0.02% formic acid; |
| Flow rate: | 1 ml/min |
| Detection: | UV 215 nm, 254 nm, 275 nm |
| | API-ES Positive |

| | RETENTION | MASS |
|---|---|---|
| INTERMEDIATE | (MIN) | (M + 1) |
| 4-(2-Oxo-4-phenyl-oxazolidin- | 3.22 | 325 |
| 3-yl)-N-propyl-benzamide. | | |
| 4-(2-Hydroxy-1-phenyl-ethylamino)- | 2.33 | 299 |
| N-propyl-benzamide | | |
| 4-(2-Oxo-4-phenyl-[1,2,3]oxathiazolidin- | 3.86 | 345 |
| 3-yl)-N-propyl-benzamide | | |
| 4-(2,2-Dioxo-4-phenyl-[1,2,3]oxathiazolidin- | 4.42 | 361 |
| 3-yl)-N-propyl-benzamide | | |
| Benzoic acid 1-[2-phenyl-2-(4-propyl | 2.38 | 472 |
| carbamoyl-phenylamino)-ethyl]-pyrrolidin- | | |
| 3-yl ester | | |
| Benzoic acid 1-{2-[methyl-(4-propyl | 2.78 | 486 |
| carbamoylphenyl)-amino]-2-phenyl-ethyl}- | | |
| pyrrolidin-3-yl ester | | |

1. A single-step or multi-step process for the preparation of a compound of formula (XI): 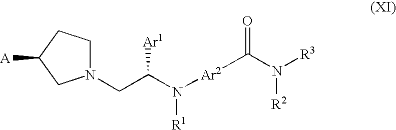
or a stereoisomer thereof, wherein;
A is hydrogen, hydroxy, C₁-C₆ (preferably C₁-C₄) alkyl, C₁-C₆ (preferably C₁-C₄) fluoroalkyl (particularly —CF₃), C₁-C₆ (preferably C₁-C₄) alkoxy, or OY wherein Y is a hydroxy protecting group or A, taken together with its geminal hydrogen, is an oxo group;
Ar¹ is phenyl optionally substituted by one or more (preferably one to two) substituents selected from fluoro, C₁-C₄ alkyl, C₁-C₄ alkoxy, C₁-C₄ alkoxy-C₁-C₄ alkoxy, trifluoromethyl, carboxy-C₁-C₄ alkoxy and C₁-C₄ alkoxycarbonyl-C₁-C₄ alkoxy;
Ar² is phenyl, naphthyl, pyridyl, thienyl, furyl, pyrrolyl or pyrimidyl, each being optionally substituted by one or more (preferably one to two) substituents selected from fluoro, C₁-C₄ alkyl, C₁-C₄ alkoxy, di(C₁-C₄)alkylamino and C₁-C₄ fluoroalkyl;
R¹ is C₁-C₆ alkyl or benzyl wherein the phenyl moiety of said benzyl is optionally substituted with C₁-C₆ alkoxy or OY wherein Y is a hydroxy protecting group; and
R² and R³ are independently selected from hydrogen, C₁-C₇ alkyl optionally substituted by one or more (preferably one to five) hydroxy or halo groups, C₃-C₆ cycloalkyl, C₂-C₆ alkenyl, C₂-C₆ alkynyl, C₁-C₇ (preferably C₁-C₅) alkoxy, phenyl optionally substituted by fluoro (preferably substituted by one or two fluoro groups), phenyl-C₁-C₇ (preferably C₁-C₅) alkyl wherein the phenyl group is optionally substituted by fluoro, and —(CH₂)ₙX—R⁴ wherein n is one or two, X is O or S and R⁴ is C₁-C₃ alkyl, or, when Ar² is phenyl, —Ar²—C(═O)—N(R²)— is a phthalimide group and R³ is C₁-C₇ alkyl; or
R² and R³, together with the nitrogen atom to which they are attached, form a pyrrolidine, piperidine or morpholine ring, optionally substituted by C₁-C₃ alkyl or fluoro;
comprising a step in which the N—Ar² bond is constructed by a copper-mediated aryl amination.

2. A process as claimed in claim 1 wherein a compound of formula (IV): 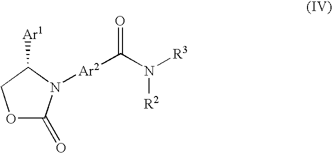
or the enantiomer thereof, wherein Ar¹, Ar², R² and R³ are as defined in claim 1, is prepared by treating a compound of formula (II): 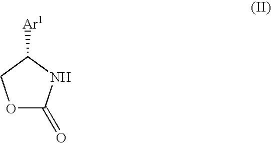
or the enantiomer thereof, wherein Ar¹ is as defined in claim 1, with a compound of formula (III): 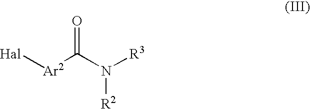
wherein Ar², R² and R³ are as defined in claim 1 and wherein one unsubstituted position on the Ar² moiety is substituted with a halogen group Hal, preferably Cl, Br or 1, most preferably Br, in the presence of a cuprous salt, an amino ligand and a base.

3. A process as claimed in claim 2 wherein the cuprous salt is CuI, CuBr or CuCl.

4. A process as claimed in claim 2 wherein the amino ligand is 1,2 diaminocyclohexane.

5. A process as claimed in claim 2 wherein the base is sodium carbonate, potassium carbonate or cesium carbonate.

6. A process as claimed in claim 1 wherein a compound of formula (V): 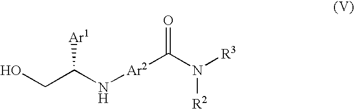
or the enantiomer thereof, wherein Ar¹, Ar², R² and R³ are as defined in claim 1, is prepared by treating a compound of formula (IV): 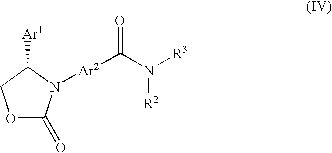
or the enantiomer thereof, wherein Ar¹, Ar², R² and R³ are as defined in claim 1, with a base in the presence of water.

7. A process as claimed in claim 1 wherein a compound of formula formula (VI): 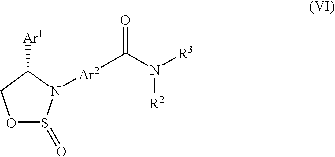
wherein Ar¹, Ar², R² and R³ are as defined in claim 1, or the enantiomer thereof, is prepared by treating a compound of formula (V): 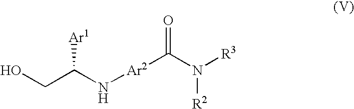
or the enantiomer thereof, wherein Ar¹, Ar², R² and R³ are as defined in claim 1, with a thionyl halide.

8. A process as claimed in claim 1 wherein a compound of formula (VII): 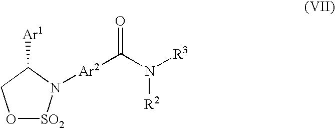
wherein Ar¹, Ar², R² and R³ are as defined in claim 1, or the enantiomer thereof, is prepared by oxidising a compound of formula (VI): 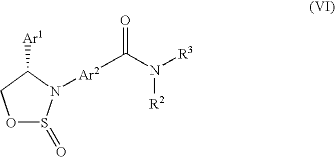
wherein Ar¹, Ar², R² and R³ are as defined in claim 1, or the enantiomer thereof.

9. A process as claimed in claim 1 wherein a compound of formula (IX): 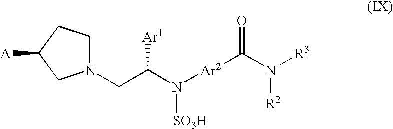
wherein A, Ar¹, Ar², R² and R³ are as defined in claim 1, or a zwitterion thereof, or a stereoisomer of either, is prepared by treating a compound of formula (VII): 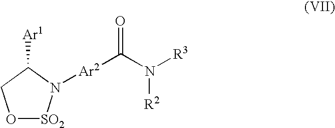
wherein Ar¹, Ar², R² and R³ are as defined in claim 1, or the enantiomer thereof, with a compound of formula (VIII): 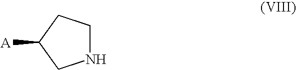
wherein A is as defined in claim 1, or the enantiomer thereof.

10. A process as claimed in claim 1 wherein a compound of formula (X): 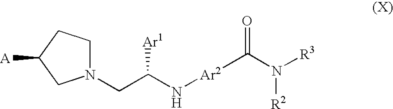
wherein A, Ar¹, Ar², R² and R³ are as defined in claim 1, or a stereoisomer thereof is prepared by hydrolytically cleaving the —SO₃H group in a compound of formula (IX): 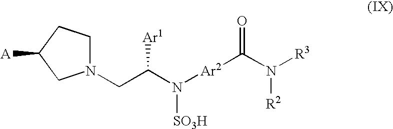
wherein A, Ar¹, Ar², R² and R³ are as defined in claim 1, or a zwitterion thereof, or a stereoisomer of either.

11. A process as claimed in claim 1 wherein a compound of the formula (XI), as defined in claim 1, or a stereoisomer thereof, is prepared by the reductive alkylation of a compound of formula (X): 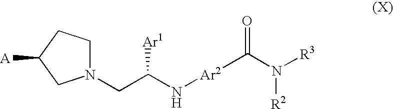
wherein A, Ar¹, Ar², R² and R³ are as defined above, or a stereoisomer thereof.

12. A process for the preparation of a compound of formula (XI), as defined in claim 1, or a stereoisomer thereof, comprising the reductive amination of a compound of formula (X): 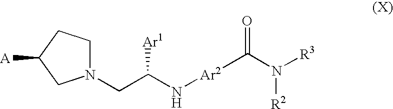
or a stereoisomer thereof, wherein A, Ar¹, Ar², R² and R³ are as defined in claim 1.

13. A process for the preparation of a compound of formula (IV): 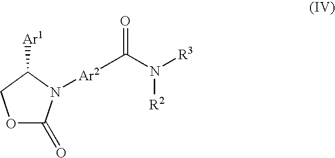
or the enantiomer thereof, wherein Ar¹, Ar², R² and R³ are as defined in claim 1, comprising treating a compound of formula (II): 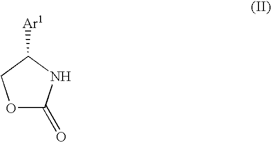
or the enantiomer thereof, wherein Ar¹ is as defined in claim 1, with a compound of formula (III): 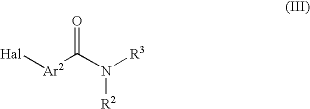
wherein Ar², R² and R³ are as defined in claim 1 and wherein one unsubstituted position on the Ar² moiety is substituted with a halogen group Hal, preferably Cl, Br or I, most preferably Br, in the presence of a cuprous salt, an amino ligand and a base.

14. A compound of formula: [chemical structure]
